(19) United States
(12) Patent Application Publication
(10) Pub. No.: US 2008/0119463 A1
(43) Pub. Date: May 22, 2008

(54) 4-Anilino-3-quinolinecarbonitriles for the treatment of acute myelogenous leukemia (AML)

(75) Inventor: Frank Boschelli, New City, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/985,100

(22) Filed: Nov. 14, 2007

Related U.S. Application Data
(60) Provisional application No. 60/859,847, filed on Nov. 16, 2006.

(51) Int. Cl. A61K 31/5375; A61K 31/435; A61K 31/47; A61P 35/02; A61K 31/495

(52) U.S. Cl. 514/231.2; 514/252.12; 514/290; 514/311

(57) ABSTRACT

The claimed invention is directed to a method of treating, inhibiting, or preventing acute myelogenous leukemia, also called acute myeloid leukemia (AML), using 4-anilino-3-quinolinecarbonitriles.

This application claims priority from copending provisional application No. 60/859,847 filed on Nov. 16, 2006 the entire disclosures of which are hereby incorporated by reference.

### FIELD OF THE INVENTION

The claimed invention is directed to a method of using 4-anilino-3-quinolinecarbonitriles to treat, inhibit, or prevent acute myelogenous leukemia, also called acute myeloid leukemia (AML).

### RELATED BACKGROUND ART

Various 4-anilino-3-quinolinecarbonitriles derivatives have been shown to have anti-neoplastic activity that may make them useful as chemoagents in treating various cancers, including pancreatic, lymphatic and prostate cancers. U.S. Pat. Nos. 6,002,008, 6,384,051, 6,432,979 and 6,617,333 disclose certain 4-anilino-3-quinolinecarbonitriles derivatives that are shown to possess anti-neoplastic activity. AML is a hematological malignancy of the myeloid line of white blood cells, characterized by the rapid proliferation of abnormal cells that accumulate in the bone marrow and interfere with the production of normal blood cells. The information presented herein demonstrates that 4-anilino-3-quinolinecarbonitriles may also be useful to treat AML.

Specific signal transducers and activators of transcription (STAT) family members are constitutively activated in various myeloid malignancies and contribute to tumor cell proliferation and resistance to apoptosis. For example, both Stat3 and Stat5 are constitutively activated in AML cells. Growth factors and cytokines, such as IL-3, GM-CSF, erythropoietin, and thrombopoietin, effect responses through JAK/STAT signaling pathways, activating primarily Stat3 and Stat5 in hematopoietic progenitor cells. Constitutive activation of STAT proteins has also been reported in cells transformed by diverse oncoproteins and tumor viruses, such as Src and Abl tyrosine kinases.

The protein tyrosine kinases consist of functionally related receptor and nonreceptor signaling enzymes regulating cell growth, activation, differentiation, development, and transformation through phosphorylation of specific tyrosine residues. The receptor tyrosine kinases, such as epidermal growth factor receptor (EGFR), consist of an extracellular ligand binding domain, a single transmembrane domain and an intracellular tyrosine kinase domain. The nonreceptor tyrosine kinases, such as Src and Abl, are soluble cytoplasmic enzymes with multiple regulatory and protein-binding domains.

The Src tyrosine kinase family is a group of 9 nonreceptor tyrosine kinases defined by both functional and sequence similarity. Three members of this family are widely expressed: Src, Yes, and FynB. The other 6 members, Lck, Lyn, FynT, Fgr, Hck, and Blk, are predominantly expressed in hematopoietic cells. Extensive reviews on structure and function of nonreceptor protein tyrosine kinases and their relevance in human cancers have been published.

The Src nonreceptor protein tyrosine kinase is the prototype of the Src family. Src is a key downstream component of pathways mediated by growth factor receptors and G-protein coupled receptors, and is believed to coordinate signals from these various pathways. The list of intracellular target proteins known to be phosphorylated by Src-family kinases is large and continues to grow, including integrins, adhesion kinases, cadherins, stat3, stat5, cortactin, ezrin, focal adhesion proteins (FAK), and many others.

Src is upregulated in most cancers, including the vast majority of hematological malignancies. Based on the above observations, compounds that inhibit Src activity may be useful in treating patients with AML.

### BRIEF SUMMARY OF INVENTION

The claimed invention is directed to a method of treating, inhibiting, or preventing AML, comprising providing a therapeutically effective amount of a compound of Formula (I):

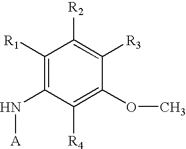

wherein:

R₁, R₂, R₃, and R₄ are each independently hydrogen, a halogen, an alkyl, or an alkoxy; and

A is

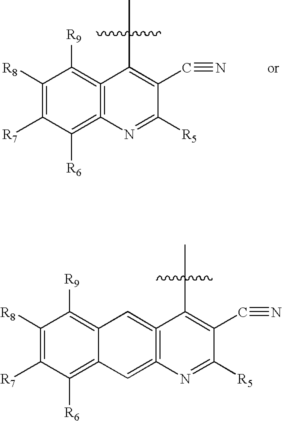

wherein R₅, R₆, and R₉ are each independently hydrogen, a halogen, an alkyl, or an alkoxy;

R₇ is —O—(CH₂)ₙ—R₁₀, -furyl-(CH₂)ₙ—R₁₀, or -pyridinyl-(CH₂)ₙ—R₁₀, wherein n is 0-3, and R₁₀ is an unsubstituted or alkyl-substituted piperazinyl or morpholinyl; and

R₈ is hydrogen or an alkoxy;

or pharmaceutically acceptable salts thereof.

This invention is also directed to a method of treating, inhibiting, or preventing AML, comprising providing a therapeutically effective amount of a compound of Formula (I), or pharmaceutically acceptable salts thereof, and a pharmaceutically acceptable carrier. The compounds of Formula (I) may be delivered alone or in combination with one or more other compounds used to treat AML.

Specific compounds of this invention, for example, include:
4-[(2,4-dichloro-5-methoxy-phenyl)amino]-6-methoxy-7-[3-(4-methyl-piperazin-1-yl)-propoxy]-quinoline-3-carbonitrile;
4-[(2,4-dichloro-5-methoxy-phenyl)amino]-6-methoxy-7-{5-[(4-methyl-piperazin-1-yl)methyl]-3-furyl}-quinoline-3-carbonitrile;
4-(2,4-dichloro-5-methoxy-anilino)-7-{5-[(4-morpholinyl)-methyl]-2-pyridinyl]-quinoline-3-carbonitrile;
4-(2,4-dichloro-5-methoxy-anilino)-7-{5-[(4-methyl-piperazin-1-yl)-methyl]-2-pyridinyl]-quinoline-3-carbonitrile;
4-(2,4-dichloro-5-methoxy-anilino)-7-methoxy-8-[2-(4-morpholinyl)-ethoxy]-benzo[g]-quinoline-3-carbonitrile;
4-(2-chloro-4 methyl-5-methoxy-anilino)-7-methoxy-8-[2-(4-morpholinyl)-ethoxy]-benzo[g]-quinoline-3-carbonitrile;
4-(3,4,5-methoxy-anilino)-7-methoxy-8-[2-(4-morpholinyl)-ethoxy]-benzo[g]-quinoline-3-carbonitrile;

and pharmaceutically acceptable salts thereof.

### DETAILED DESCRIPTION OF THE INVENTION

This invention is directed to a method of treating, inhibiting, or preventing AML comprising providing a therapeutically effective amount of a compound of Formula (I), or pharmaceutically acceptable salts thereof.

For the purposes of this invention the term “inhibiting” refers to retarding, suppressing, or stopping malignant cell proliferation, presumably by blocking or suppressing phosphorylation catalyzed by Src. For the purposes of this invention the term “preventing” refers to averting or forestalling the development of malignant or tumoric growths by prophylactic treatment, or to impede, inhibit, or cease further progression of the disease. For the purposes of this invention, the term “therapeutically effective amount” refers to an amount of compound sufficient to cure, inhibit, or ameliorate symptoms of AML. For purposes of this invention the term “alkyl” includes both straight and branched alkyl moieties, preferably having 1 to 8 carbons. For purposes of this invention the term “alkoxy” is defined as alkyl-O—. As used herein, halogen may be selected from chloride, bromide and fluoride moieties. As used herein, “alkyl-substituted piperazinyl” or “alkyl-substituted morpholinyl” means the nitrogen of the piperazinyl moiety may be substituted with an alkyl moiety or one or more of the ring carbons of either piperazinyl or morpholinyl may be substituted with an alkyl moiety.

A compound of Formula (I) may be provided orally, topically, by intralesional, intraperitoneal, intramuscular or intravenous injection, by infusion, or by nasal, anal, vaginal, sublingual, uretheral, transdermal, intrathecal, ocular, otic, or liposome-mediated delivery. In order to obtain consistency in providing a compound of Formula (I), it is preferred that the compound is in the form of the unit dose. Suitable unit dose forms include tablets, capsules, and powders, in sachets or vials. Such unit dose forms may contain from about 0.1 to about 300 mg of a compound of Formula (I), and preferably from about 2 to about 200 mg. Still further preferred unit dosage forms contain about 50 to about 150 mg of a compound of Formula (I). A compound of Formula (I) may be administered from 1 to 6 times a day, and more usually from 1 to 4 times a day. The effective amount will be known to one of skill in the art, and will depend upon the form of the compound, the purpose of administration, and the like. One of skill in the art could routinely perform empirical activity tests to determine the bioactivity of a compound of Formula (I) in bioassays and thus determine what dosage to administer.

Pharmaceutically acceptable salts, for example, are those derived from such organic and inorganic acids as: acetic, lactic, carboxylic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

This invention is also directed to a method of treating, inhibiting, or preventing AML, comprising providing a therapeutically effective amount of a compound of Formula (I), or pharmaceutically acceptable salts thereof, and a pharmaceutically acceptable carrier. The carrier may be, for example, a diluent, an aerosol, a topical carrier, an aqueous solution, a nonaqueous solution, or a solid. The carrier may also be a polymer or a toothpaste. A carrier in this invention encompasses any of the standard pharmaceutically accepted carriers, such as water, phosphate buffered saline (PBS) solution, acetate buffered saline solution, emulsions, such as an oil/water emulsion or a triglyceride emulsion, various types of wetting agents, tablets, coated tablets and capsules. The compositions containing the compound of Formula (I) may be formulated with conventional excipients, such as fillers, disintegrating agents, binders, lubricants, flavoring agents, or color additives.

When provided orally or topically, such compounds would be provided to a subject by delivery in different carriers. Typically, such carriers contain excipients such as starch, milk, sugar, certain types of clay, gelatin, stearic acid, talc, vegetable fats or oils, gums, or glycols. The specific carrier would need to be selected based upon the desired method of delivery. For example, PBS could be used for intravenous or systemic delivery, and vegetable fats, creams, salves, ointments, or gels may be used for topical delivery.

A compound of Formula (I) may be delivered together with suitable diluents, preservatives, solubilizers, emulsifiers, adjuvants and/or carriers useful in treatment or prevention of neoplasm. Such compositions are liquids or lyophilized or otherwise dried formulations, and include diluents of various buffer content (for example, Tris-HCl, acetate, or phosphate) and pH and ionic strength, additives such as albumins or gelatin to prevent absorption to surfaces, detergents (for example, TWEEN 20, TWEEN 80, PLURONIC F68, or bile acid salts), solubilizing agents (for example, glycerol or polyethylene glycerol), anti-oxidants (for example ascorbic acid or sodium metabisulfate), preservatives (for example, thimerosal, benzyl alcohol or parabens), bulking substances or tonicity modifiers (for example, lactose or mannitol), covalent attachment of polymers such as polyethylene glycol, complexation with metal ions, or incorporation of the compound into or onto particulate preparations of hydrogels, liposomes, micro-emulsions, micelles, unilamellar or multilamellar vesicles, erythrocyte ghosts, or spheroblasts. Such compositions will influence the physical state, solubility, stability, rate of in vivo release, and rate of in vivo clearance of the compound or composition. The choice of compositions will depend on the physical and chemical properties of the compound capable of treating or preventing a neoplasm.

The compound of Formula (I) may be delivered locally via a capsule that allows a sustained release of the compound over a period of time. Controlled or sustained release compositions include formulation in lipophilic depots (for example, fatty acids, waxes, or oils).

The present invention further provides a method of using a compound of Formula (I) as an active therapeutic substance for treating, inhibiting, or preventing AML.

The compound of Formula (I) may be delivered alone or in combination with other compounds used to treat AML. Such compounds include but are not limited to daunorubicin, cytarabine, thioguanine, idarubicin, methotrexate, all-trans retinoic acid, mercaptopurine, mylotarg, etoposide, arsenic trioxide, and mitoxantrone.

Preferred compounds for practicing the method of and/or for use in a composition of this invention are 4-[(2,4-dichloro-5-methoxy-phenyl)amino]-6-methoxy-7-[3-(4-methyl-piperazin-1-yl)-propoxy]-quinoline-3-carbonitrile and pharmaceutically acceptable salts thereof.

The compounds of Formula (I) may be prepared according to the methods disclosed in U.S. Pat. Nos. 6,002,008 and 6,780,996, and such methods are hereby incorporated by reference.

Reactions are performed in a solvent appropriate to the reagents and materials employed and suitable for the transformation being effected. It is understood by those skilled in the art of organic synthesis that the various functionalities present on the molecule must be consistent with the chemical transformations proposed. When not specified, order of synthetic steps, choice of protecting groups, and deprotection conditions will be readily apparent to those skilled in the art. In addition, in some instances, substituents on the starting materials may be incompatible with certain reaction conditions. Restrictions pertinent to given substituents will be apparent to one skilled in the art. Reactions are run under inert atmospheres where appropriate.

The following experimental details are set forth to aid in an understanding of the invention, and are not intended, and should not be construed, to limit in any way the invention set forth in the claims that follow thereafter.

Table 1 shows proliferation assay results obtained upon treating human AML cell lines with several 4-anilino-3-quinolinecarbonitriles of Formula (I). These compounds were prepared according to previously published methods (Boschelli, D. H., et. al., J. Med. Chem., 44, 3965 (2001); Boschelli, D. H., et. al., J. Med. Chem., 44, 822 (2001); Boschelli, D. H., et. al., Bioorg. Med. Chem. Lett., 13, 3797 (2003); Boschelli, D. H., et. al., J. Med. Chem., 47, 1599 (2004); and Ye, F. et al., 221^{st} National Meeting of the American Chemical Society, San Diego, Calif. (April, 2001)), hereby incorporated by reference. For this study, 1×10² to 1×10³ cells were plated in 95 μl of growth medium in each well of a 96-well microtiter plate. Compounds were added in 5 μl of medium such that the final concentration of DMSO in the medium was 0.25%. Cells were allowed to grow in a cell culture incubator (37° C.) for 3 days, at which time 100 μl of Promega Cell Titer Glo agent was added. Luminescence (lum) was measured in an Envision microplate reader. Data were processed by first obtaining % inhibition (100−(lum+cpd/lum−cpd); cpd refers to cycles per degree). The data were then analyzed with the LSW IC₅₀ calculation model ligand-receptor binding/hyperbolic (y=Bmax/(1+(x/IC₅₀)). The IC₅₀ is the concentration of test compound needed to reduce the total amount of cell proliferation by 50%.

Based upon the results obtained and presented herein, 4-anilino-3-quinolinecarbonitriles are useful in treating, inhibiting, or preventing AML by suppressing proliferation of malignant cells, at least in part by inhibiting Src-catalyzed phosphorylation of cellular proteins. Therefore, administration of a therapeutically effective amount of a compound of Formula (I) may prevent or inhibit AML by suppressing malignant cell proliferation, or may treat a human already suffering from AML by preventing or inhibiting further progression of the disease.

TABLE 1
IC₅₀ (μM) values for 4-anilino-3-quinolinecarbonitriles from
proliferation assays of human AML cells lines.

| | IC₅₀ |
|---|---|
| | (μM) |
| U937 | |
| 4-[(2,4-dichloro-5-methoxy-phenyl)amino]-6-methoxy-7-[3-(4- | 1.26 |
| methyl-piperazin-1-yl)-propoxy]-quinoline-3-carbonitrile | 3.8 |
| 4-[(2,4-dichloro-5-methoxy-phenyl)amino]-6-methoxy-7-{5-[(4- | 0.25 |
| methyl-piperazin-1-yl)methyl]-3-furyl}-quinoline-3-carbonitrile | 0.91 |
| 4-(2,4-dichloro-5-methoxy-anilino)-7-{5-[(4-methyl-piperazin- | 2.4 |
| 1-yl)-methyl]-2-pyridinyl]-quinoline-3-carbonitrile | |
| 4-(3,4,5-methoxy-anilino)-7-methoxy-8-[2-(4-morpholinyl)- | 2.1 |
| ethoxy]-benzo[g]-quinoline-3-carbonitrile | |
| 4-(2,4-dichloro-5-methoxy-anilino)-7-methoxy-8-[2-(4- | 5.1 |
| morpholinyl)-ethoxy]-benzo[g]-quinoline-3-carbonitrile | |
| 4-(2-chloro-4 methyl-5-methoxy-anilino)-7-methoxy-8-[2-(4- | 1.2 |
| morpholinyl)-ethoxy]-benzo[g]-quinoline-3-carbonitrile | |
| 4-(2,4-dichloro-5-methoxy-anilino)-7-{5-[(4-morpholinyl)-methyl]- | 0.73 |
| 2-pyridinyl]-quinoline-3-carbonitrile; | |
| KG-1a | |
| 4-[(2,4-dichloro-5-methoxy-phenyl)amino]-6-methoxy-7-[3-(4- | 1.11 |
| methyl-piperazin-1-yl)-propoxy]-quinoline-3-carbonitrile | |
| 4-[(2,4-dichloro-5-methoxy-phenyl)amino]-6-methoxy-7-{5-[(4- | 0.78 |
| methyl-piperazin-1-yl)methyl]-3-furyl}-quinoline-3-carbonitrile | |

For relevant experiments described above, standard growth medium for a particular cell line was used. For example, the growth medium for U937 cells is: RPMI 1640 supplemented with glutamine, 10% fetal bovine serum, and 50 μg/ml of gentamicin.

Therefore, the compounds described herein may represent a novel therapy for AML.

What is claimed is:

1. A method of treating, inhibiting, or preventing AML comprising, providing a therapeutically effective amount of a compound of Formula (I): 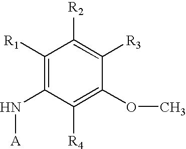
wherein:
R₁, R₂, R₃, and R₄ are each independently hydrogen, a halogen, an alkyl, or an alkoxy; and
A is 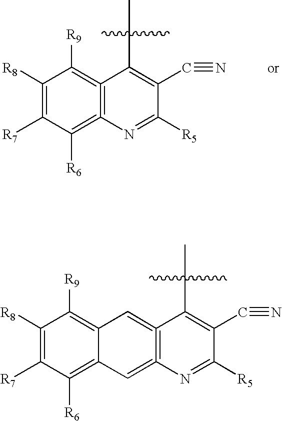
wherein R₅, R₆, and R₉ are each independently hydrogen, a halogen, an alkyl, or an alkoxy;
R₇ is —O—(CH₂)ₙ—R₁₀, -furyl-(CH₂)ₙ—R₁₀, or -pyridinyl-(CH₂)ₙ—R₁₀, wherein n is 0-3, and R₁₀ is an unsubstituted or alkyl-substituted piperazinyl or morpholinyl; and
R₈ is hydrogen or an alkoxy;
or a pharmaceutically acceptable salt thereof.

2. The method of claim 1, wherein R₁ and R₃ are Cl.

3. The method of claim 2, wherein R₂, R₄, R₅, R₆, and R₉, are hydrogen.

4. The method of claim 3, wherein R₈ is methoxy.

5. The method of claim 4, wherein m is 0, R₇ is —O—(CH₂)ₙ—R₁₀, n is 3, and R₁₀ is an alkyl-substituted piperazine.

6. The method of claim 1, wherein the compound of Formula (I) is: 4-[(2,4-dichloro-5-methoxy-phenyl)amino]-6-methoxy-7-[3-(4-methyl-piperazin-1-yl)-propoxy]-quinoline-3-carbonitrile; 4-[(2,4-dichloro-5-methoxy-phenyl)amino]-6-methoxy-7-{5-[(4-methyl-piperazin-1-yl)methyl]-3-furyl}-quinoline-3-carbonitrile; 4-(2,4-dichloro-5-methoxy-anilino)-7-{5-[(4-morpholinyl)-methyl]-2-pyridinyl]-quinoline-3-carbonitrile; 4-(2,4-dichloro-5-methoxy-anilino)-7-{5-[(4-methyl-piperazin-1-yl)-methyl]-2-pyridinyl]-quinoline-3-carbonitrile; 4-(2,4-dichloro-5-methoxy-anilino)-7-methoxy-8-[2-(4-morpholinyl)-ethoxy]-benzo[g]-quinoline-3-carbonitrile; 4-(2-chloro-4 methyl-5-methoxy-anilino)-7-methoxy-8-[2-(4-morpholinyl)-ethoxy]-benzo[g]-quinoline-3-carbonitrile; or 4-(3,4,5-methoxy-anilino)-7-methoxy-8-[2-(4-morpholinyl)-ethoxy]-benzo[g]-quinoline-3-carbonitrile.

7. The method of claim 1, wherein the compound of Formula (I) is 4-[(2,4-dichloro-5-methoxy-phenyl)amino]-6-methoxy-7-[3-(4-methyl-piperazin-1-yl)-propoxy]-quinoline-3-carbonitrile.

8. The method of claim 1, wherein the compound of Formula (I) is delivered alone or in combination with one or more other compounds used to treat AML.

9. A method of treating, inhibiting, or preventing AML comprising, providing a therapeutically effective amount of a compound of Formula (I): 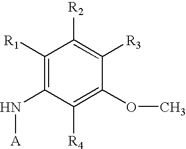
wherein:
R₁, R₂, R₃, and R₄ are each independently hydrogen, a halogen, an alkyl, or an alkoxy; and
A is 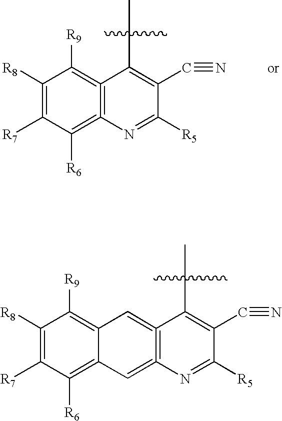
wherein R₅, R₆, and R₉ are each independently hydrogen, a halogen, an alkyl, or an alkoxy;
R₇ is —O—(CH₂)ₙ—R₁₀, -furyl-(CH₂)ₙ—R₁₀, or -pyridinyl-(CH₂)ₙ—R₁₀, wherein n is 0-3, and R₁₀ is an unsubstituted or alkyl-substituted piperazinyl or morpholinyl; and
R₈ is an alkoxy;
or a pharmaceutically acceptable salt thereof.
